(19) United States
(12) Patent Application Publication
(10) Pub. No.: US 2006/0129121 A1
(43) Pub. Date: Jun. 15, 2006

(54) Absorbent article with large stretch fastener

(76) Inventor: Carol Erdman, West Chester, PA (US)

Correspondence Address: Mark S. Leonardo, Esq.;Brown Rudnick Berlack Israels LLP, One Financial Center, Boston, MA, 02111, US

(21) Appl. No.: 11/010,803

(22) Filed: Dec. 13, 2004

(51) Int. Cl. A61F 13/15

(52) U.S. Cl. 604/389; 604/391

(57) ABSTRACT

A fastener is provided that is adapted for use with an absorbent article. The fastener includes a rectangular extensible portion configured to extend in a lateral direction such that the fastener extends distally from the absorbent article. The extensible portion of the fastener includes a fastener attachment region for attachment along a distal edge of the absorbent article. The fastener attachment region is located distally of a proximal edge of the extensible portion. The fastener further includes a non-extensible closure tab portion. The non-extensible closure tab portion has a closure tab attachment region for attachment along a distal edge of the extensible portion. The closure tab attachment region is located distally from a proximal edge of the non-extensible portion. The non-extensible portion of the fastener may be substantially centered along a distal edge of the fastener's extensible portion.

### BACKGROUND

1. Technical Field

The present disclosure generally relates to absorbent articles, and more particularly, to disposable absorbent articles with improved fasteners.

2. Background of the Related Art

Absorbent articles such as, for example, disposable diapers, adult incontinent pads, sanitary napkins, pantiliners, incontinent garments, etc. are generally worn, in cooperation with garments and disposed against a body surface by infants or adult incontinent individuals. The absorbent article is employed to collect and absorb body fluid discharge, such as, for example, blood, menses, urine, aqueous body fluids, mucus and cellular debris. For example, the absorbent article may be disposed between the legs of an individual adjacent a crotch area. The absorbent article is positioned with a garment and drawn into engagement with a body surface of the crotch area to collect fluid discharge.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as, for example, wood pulp, for acquiring and storing fluid discharge.

In addition, fastening of the absorbent articles with an individual requires the use of fasteners or ear members and closure tabs that extend laterally from the body of the absorbent article. In addition, the closure tabs typically include mechanical closure material, for example, hook and/or loop material, adhesive tape or the like. For example, in typical diaper-type garments, the garment is affixed to a wearer by attaching one or more of the closure tabs that extend across the wearer's hips to hold the back and front halves of the garment to one another.

Some of these absorbent articles, however, suffer from performance drawbacks including that fastening of the articles resulting in poor fit and waste containment.

It would therefore be desirable to overcome the disadvantages and drawbacks of the prior art by providing an absorbent article including improved fasteners.

### SUMMARY

Accordingly, an absorbent article is disclosed that includes an absorbent article with a large stretch closure system. Objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure consisting of its constituent parts, constructions, arrangements, combinations, steps and improvements herein shown and described.

In one particular embodiment, the fastener is adapted for use with an absorbent article. The fastener includes a rectangular extensible portion configured to extend in a lateral direction such that the fastener extends distally from the absorbent article. The extensible portion of the fastener includes a fastener attachment region for attachment along a distal or most distal edge of the absorbent article. The fastener attachment region is located distally of a proximal edge of the extensible portion. The fastener further includes a non-extensible closure tab portion. The non-extensible closure tab portion has a closure tab attachment region for attachment along a distal edge of the extensible portion. The closure tab attachment region is located distally from a proximal edge of the non-extensible portion. The non-extensible portion of the fastener may be substantially centered along a distal edge of the fastener's extensible portion. The non-extensible portion may include hook elements configured for attachment with the absorbent article. The extensible portion of the fastener may be disposed between the absorbent article and the fastener's non-extensible portion. Alternatively, the extensible portion has a greater surface area relative to the non-extensible portion.

In an alternative embodiment, an absorbent article includes a backsheet, a topsheet and an absorbent core disposed therebetween. The absorbent article extends from a first end to a second end along a longitudinal length thereof. A fastener is attached to the first end engageable with the second end for disposing the absorbent article about a subject. The first end includes a fastener attachment area for attaching the fastener, wherein a length of the fastener is between 50-90% of a length of the fastener attachment area. (Alternatively, the length of the fastener attachment area may be in the range of 100-130 mm, and the length of the fastener can be in the range of 50-120 mm.

A closure tab may be attached to the fastener for engaging the second end of the absorbent article, wherein the length of the closure tab is between 50 and 90 percent of the length of the fastener.

In another embodiment, the fastener may be rectangular and include a distal non-stretch end, a proximal non-stretch end, and a stretch section between the non-stretch ends. A rectangular non-stretch closure tab has a proximal end and a distal end. Mechanical closure means are attached to the closure tab and engageable with the second end for disposing the absorbent article about the subject. The proximal non-stretch end of the fastener is attached to a most distal edge of the front end such that the fastener is substantially centered along this most distal edge. The distal non-stretch end of the fastener is attached to the proximal end of the closure tab such that the closure tab is substantially centered along the distal non-stretch end of the fastener. The distal end of the closure tab is attached to the mechanical closure means.

The proximal non-stretch end of the fastener may overlap the most distal edge of the front end in the range of 20-30 mm. The distal non-stretch end of the fastener overlaps the proximal end of the closure tab front end in the range of 10-30 mm. The proximal non-stretch end of the fastener can include a region for attachment with the front end, the region for attachment having a width in the range of 5-20 mm. The region for attachment may be disposed from a distal edge of the fastener by a distance in the range of 1-4 mm.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1A is a plan view of an absorbent article according to the present disclosure;

FIG. 1B is a partial side view of the absorbent article shown in FIG. 1A;

FIG. 2A is an exploded plan view illustrating a cutaway portion and a fastener of the absorbent article shown in FIG. 1A;

FIG. 2B is an exploded side view of the fastener shown in FIG. 2A;

FIG. 3A is an exploded side view illustrating the cutaway portion and the fastener of the absorbent article shown in FIG. 1A;

FIG. 3B is a plan view illustrating a cutaway portion and the fastener absorbent article shown in FIG. 1A;

FIG. 4A is a side view of an alternate embodiment of the fastener; and

FIG. 4B is a side view of an alternate embodiment of the fastener.

### DETAILED DESCRIPTION OF EXEMPLARY EMBODIMENTS

The exemplary embodiments of the absorbent article and methods of use disclosed are discussed in terms of fluid absorbent articles, and more particularly, in terms of an absorbent article including fasteners that cooperate to improve attachment and fit. The presently disclosed absorbent article is contemplated to be employed with, for example, disposable diapers, training pants, adult incontinent products and other incontinent garments.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. As used herein, the term “absorbent article,” “absorbent garment” or “garment” refers to absorbent articles that absorb and contain body liquids, discharge and waste, and more specifically, refers to absorbent articles that are placed against or in proximity to the body of the wearer to absorb and contain the various body liquids, discharge and waste.

A non-exhaustive list of examples of absorbent articles includes diapers, diaper covers, disposable diapers, training pants, feminine hygiene products and adult incontinence products. The term absorbent article includes all variations thereof, including disposable absorbent articles that are intended to be discarded or partially discarded after a single use and unitary disposable absorbent articles that have essentially a single structure. As used herein, the term “diaper” refers to an absorbent article generally worn by children and incontinent persons about the lower torso.

The claims are intended to cover all of the foregoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles, including those described above.

Absorbent articles and diapers may have a number of different constructions. In each of these constructions it is generally the case that an absorbent core is disposed between a liquid pervious, body-facing topsheet and a liquid impervious, exterior facing backsheet. In some cases, one or both of the topsheet and backsheet may be shaped to form a pant-like garment. In other cases, the topsheet, backsheet and absorbent core may be formed as a discrete assembly that is placed on a main chassis layer and the chassis layer is shaped to form a pant-like garment. The garment may be provided to the consumer in the fully assembled pant-like shape or may be partially pant-like and require the consumer to take the final steps necessary to form the final pant-like shape, such as by fastening one or more fasteners or fastener tabs.

In the case of some diapers and most adult incontinent products, the garment often is provided fully formed with factory-made side seams and the garment is donned by pulling it up the wearer's legs. In the case of most diapers, wherein, for example, a child lies on his or her back, a caregiver usually places the diaper between the child's legs, pulls the front end of the diaper up between the legs and then attaches one or more closure tabs to the front waist region of the diaper, thereby forming a pant-like structure. For clarity, the present invention is described herein only with reference to a diaper-type garment in which the topsheet, backsheet and absorbent core are assembled into a structure that forms a pant-like garment when secured on a wearer using fastening devices, although the invention may be used with any other type of absorbent garment that may benefit from the use or addition of fastener tabs.

The following discussion includes a description of the absorbent article in accordance with the present disclosure. Reference will now be made in detail to the exemplary embodiments of the disclosure, which are illustrated in the accompanying FIGURES.

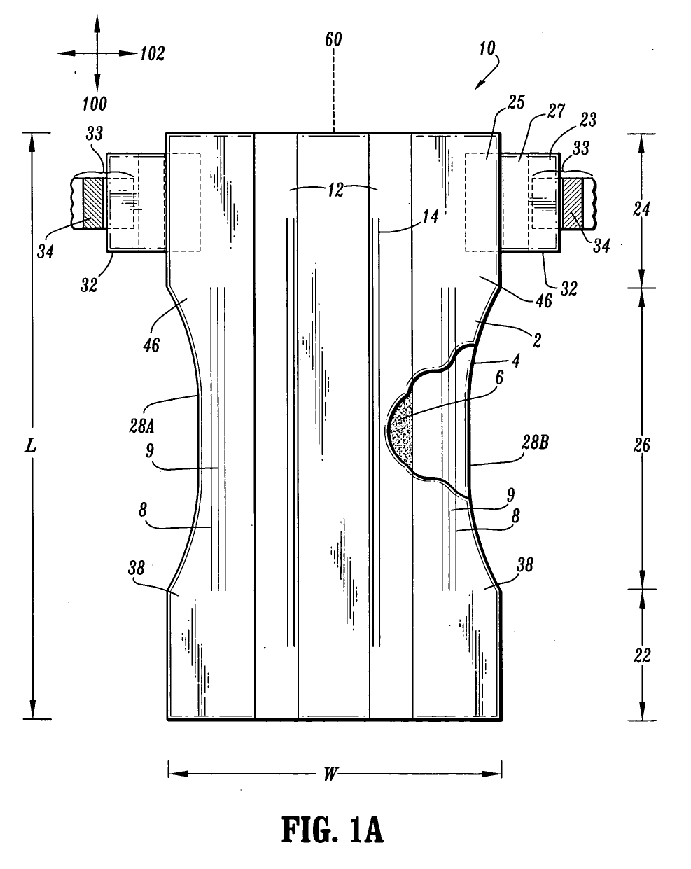

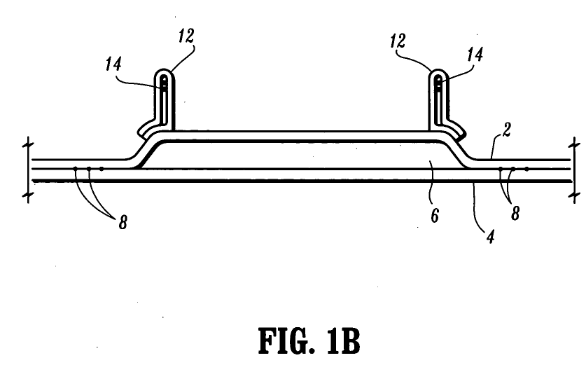

Turning now to the FIGURES, wherein like components are designated by like reference numerals throughout the several views. Referring to FIGS. 1-4, there is illustrated an absorbent article, constructed in accordance with the principles of the present disclosure, such as, for example diaper 10. In FIG. 1A, diaper 10 is shown in a relaxed condition with the effects of the elastics 8, 14 removed for purposes of clarity in the description. The diaper 10 chassis generally has an hourglass shape. The chassis generally can be defined in terms of a front waist region 22, a back waist region 24 and a crotch region 26. Those skilled in the art will recognize that “front” and “back” are relative terms, and these regions may be transposed without departing from the scope of the present invention. Alternatively, the diaper chassis can be configured in a generally rectangular shape or in a “T” shape. The diaper comprises a topsheet 2, a backsheet 4, which may be either a different size than the topsheet 2 or may be substantially coterminous with the topsheet 2 and an absorbent core 6 disposed between at least a portion of the topsheet 2 and backsheet 4. Throughout this description, the terms “topsheet” and “backsheet” denote the relationship of these materials or layers with respect to the absorbent core 6. It is understood that additional layers may be present between or beyond the absorbent core 6 and the topsheet 2 and backsheet 4 and that additional layers and other materials may be present on the side opposite the absorbent core 6 from either the topsheet 2 or the backsheet 4. A pair of leg openings 28A, 28B extend along at least a portion of the crotch region 26 and one or more pairs of leg elastics 8 (three pairs are shown in FIG. 1A) may be disposed to extend adjacent to leg openings 28a, 28b. Of course, in other embodiments, the number of leg elastics 8 may be increased, decreased or omitted altogether.

The diaper 10 generally has a longitudinal direction 100 that extends generally parallel to the front-to-back axis of a wearer, and a lateral direction 102 that extends generally parallel to the side-to-side axis of a wearer. The diaper 10 generally is symmetrical about a longitudinal centerline 60, but also may have asymmetrical components or shapes. The terms “inward” or “proximal,” and “outward” or “distal,” as used herein, refer to positions generally along the lateral direction 102, with “inward” locations being located closer to the longitudinal centerline 60 than “outward” locations.

The diaper may further include a waste containment system in the form of waste containment flaps 12 (also known as inner leg gathers (“ILG's”), unitary leg gathers or standing leg gathers) and one or more pairs of elastics 14. Waste containment flaps 12 extend from the front waist region 22 to the back waist region 24 along opposite sides of the longitudinal center line 60 of the diaper 10 or alternatively only along a portion thereof. The front waist region 22 and rear waist region 24 may include side panels or ear portions 38, 46, extending outward from the leg openings 28A, 28B to provide the garment 10 with an hourglass shape.

A variety of backsheet and topsheet constructions and materials are available and known in the art and the invention is not intended to be limited to any specific materials or constructions of these components. The backsheet 4 may be made from any suitable pliable liquid-impervious material known in the art. Typical backsheet materials include films of polyethylene, polypropylene, polyester, nylon and polyvinyl chloride and blends of these materials. For example, the backsheet can be comprised of a pigmented polyethylene film having a thickness in the range of 0.02-0.04 mils. The moisture-pervious topsheet 2 can be made of any suitable relatively liquid-pervious material known in the art that permits passage of liquid therethrough. Non-woven topsheet materials are exemplary because such materials readily allow the passage of liquids to the underlying absorbent core 6. Examples of suitable topsheet materials include non-woven spunbond or carded webs of polypropylene, polyethylene, nylon, polyester and blends of these materials.

The backsheet 4 and the topsheet 2 may be “associated” with one another. The term “associated” encompasses configurations whereby the topsheet 2 is directly joined to the backsheet 4 by affixing the topsheet 2 directly to the backsheet 4, and configurations whereby the topsheet 2 is indirectly joined to the backsheet 4 by affixing the topsheet 2 to intermediate members which in turn are affixed to the backsheet 4. While the backsheet 4 and topsheet 2 in the present disclosure have substantially the same dimensions, they may also have different dimensions.

In addition, the backsheet 4 may be covered with a fibrous, non-woven fabric layer. Materials for such a fibrous outer liner include a spun-bonded non-woven web of synthetic fibers such as polypropylene, polyethylene or polyester fibers; a non-woven web of cellulosic fibers, textile fibers such as rayon fibers, cotton and the like, or a blend of cellulosic and textile fibers; a spun-bonded non-woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers.

The backsheet 4 may comprise multiple panels, such as, for example, three panels wherein a central poly backsheet panel is positioned adjacent the absorbent core while distal non-woven breathable side backsheet panels are attached to the side edges of the central poly backsheet panel. The backsheet may also be formed from microporous poly coverstock for added breathability. In other embodiments, the backsheet may be a laminate of several sheets. The backsheet may further be treated to render it hydrophilic or hydrophobic, and may have one or more visual indicators associated with it, such as labels indicating the front or back of the diaper or other characters or colorations. The present invention is not limited to any particular backsheet 4 material or construction.

The topsheet 2 may be formed from one or more panels of material and may comprise a laminated sheet construction. As shown in FIG. 1A, the topsheet 2 comprises one panel across the entire width of the diaper and is formed from a liquid-pervious material that is either hydrophobic or hydrophilic. Topsheet 2 may be made from any number of materials, including synthetic fibers (e.g., polypropylene or polyester fibers), natural fibers (e.g., wood or cellulose), apertured plastic films, reticulated foams, porous foams, etc. Topsheet 2 may be fabricated from a cover stock of single ply non-woven material which may be made of carded fibers, either adhesively or thermally bonded, perforated plastic film, spunbonded fibers, or water entangled fibers, which generally weigh, for example, from 0.3-0.7 oz./yd² and have appropriate and effective machine direction and cross-machine direction strength suitable for use as a baby diaper cover stock material, as are known in the art. Topsheet 2 may extend from substantially the front waist region 22 to the back waist region 24 or a portion thereof, and may include a surfactant (at least along the core area) to aid in the passage of body waste to the core 6.

As shown in FIGS. 1A and 1B, the waste containment flaps 12 may be formed from separate elasticized strips of material that are associated with the topsheet 2, backsheet 4 or both, or otherwise integrated into the garment. The topsheet 2 and backsheet 4 may have similar dimensions or different dimensions, but in either case, the waste containment flaps 12 are attached to the topsheet 2 or to some intermediate element which in turn is attached to the topsheet 2. The waste containment flaps 12 may be treated with a suitable surfactant to modify their hydrophobicity or hydrophilicity or imbued with skin wellness products as desired. Each waste containment flap 12 may include a portion that folds over onto itself to form an enclosure. One or more elastic members 14 may be secured in the enclosure in a stretched condition. Various other configurations of topsheets 2 and waste containment systems, such as flaps 12, are known in the art, and the present invention is not intended to be limited to any particular design for these components.

Each leg opening 28A, 28B may be provided with a leg elastic containment system 9, sometimes referred to as conventional leg gathers. In an illustrative embodiment, three strands of elastic threads 8 are positioned to extend adjacent each leg openings 28A, 28B between the topsheet 2 and the backsheet 4. The selection of appropriate elastics and the construction of leg elastic containment systems are known in the art. For example, the leg elastics 8 may be ultrasonically bonded, heat and pressure sealed using a variety of bonding patterns, or glued to the diaper 10. Leg gathers 9 may be formed with or as separate members from topsheet 2.

Various commercially available materials may be used for the leg elastics 8 and elastic members 14, such as natural rubber, butyl rubber or other synthetic rubber, urethane, elastomeric materials such as spandex, which is marketed under various names, including LYCRA® (DuPont), GLOSPAN™ (Globe) and SYSTEM 7000™ (Fulflex). The present invention is not limited to any particular elastic material or to any particular shape, size or number of elastics.

The underlying structure beneath the topsheet 2 may include, depending on the absorbent article construction, various combinations of elements, but in each embodiment, it is contemplated that the absorbent article will include an absorbent core 6. Although the absorbent core 6 depicted in the FIGURES has a substantially rectangular shape in the plan view, other shapes may be used, such as a “T” shape or an hourglass shape. The absorbent core 6 may extend into either or both of the front and back waist regions 22, 24. The shape and construction of the absorbent core 6 may be selected to provide the greatest absorbency in target areas where body fluids are most likely to strike the diaper 10, which is often referred to as zoned absorbency. The absorbent core 6 may also comprise a number of layers of similar or different construction. The absorbent core may be associated with the topsheet 2, backsheet 4 or any other suitable part of the garment 10 by any method known in the art, in order to fix the absorbent core 6 in place.

The absorbent core 6 may include particles of super absorbent material (SAP) distributed within a fibrous structure. Additional fibrous or particulate additives may be disposed within the absorbent core 6 to add to the core's strength and SAP efficiency or to otherwise enhance the performance of the garment. The absorbent core 6 may be partially or wholly surrounded by additional layers (not shown) added to provide further benefits. The additional layer or layers may comprise any useful layer known in the art or developed hereafter, such as a fluid acquisition layer, a distribution layer, an additional fibrous layer optionally containing super absorbent particles, a wicking layer, a storage layer, or combinations and fragments of these layers. Such layers may be provided to assist with transferring fluids to the absorbent core 6, handling fluid surges, preventing rewet, containing absorbent material, improving core stability or for other purposes.

The absorbent core 6 may be made from any absorbent material or materials, or combinations of such materials, known in the art. The absorbent core 6 may include wood fibers or other fibers such as chemical wood pulp, fibrous absorbent gelling material or any other suitable liquid absorbing material, such as commercially available fluff pulp or fluffed bleached kraft softwood pulp or fibrous absorbent gelling material. Alternatively, the absorbent core 6 may include a combination of a porous fibrous web and super absorbent particles. The absorbent core may be thin to improve the comfort and appearance of a garment.

Diaper 10 is fastened onto a wearer by using fasteners 32. Fasteners 32 are affixed to the chassis of the diaper 10 to extend laterally outward (i.e., in the lateral direction 102) from waist regions 22, 24. Fasteners 32 are positioned to extend outward from the ear portions 46 of the rear waist region 24. However, the fasteners 32 may also be attached to extend outward from the front waist region 22 or from both waist regions 22, 24. The fasteners 32 may extend from one or both lateral sides of the diaper 10.

Fasteners 32 are positioned between topsheet 2 and backsheet 4. Each of a pair of closure tabs 33 is attached along distal section 23 of fastener 32, as described below in greater detail. Closure tab 33 operates by adhering to a corresponding surface, landing zone or object located on the opposite end of the diaper 10, such as, for example, front waist region 24. Alternatively, the fasteners 32 may be attached to any part of the diaper chassis, such as topsheet 2, backsheet 4, outer cover or other layer of the diaper. The fasteners 32 may also be attached to either side of the diaper's chassis, to multiple layers of the chassis, or may be sandwiched between the various sheets or layers comprising the chassis of the diaper 10.

Rectangular fastener 32 is large with respect to the smaller non-stretch closure tab 33. Fastener 32 may be rectangular, with sides of equal distance. Fastener 32 has a “zoned” stretch property, in that, only certain portions or zones thereof will extend or stretch during use. Fastener 32 includes center, distal, and proximal sections. Center section 27 is stretchable in lateral direction 102. Distal section 23 is not stretchable. Proximal section 25 is not stretchable and is attached to the diaper chassis. Fastener 32 has a deadened zone, distal section 23, proximal 25 on each side that is generally between 10-30 mm and preferably 20 mm. It is contemplated that the various fastener sections may be formed with materials that are elastic, non-elastic or a combination thereof. As described below, fastener 32 may be attached to the diaper 10 or closure tab 33 or both by adhesive, ultrasonic, thermal bonding or the like.

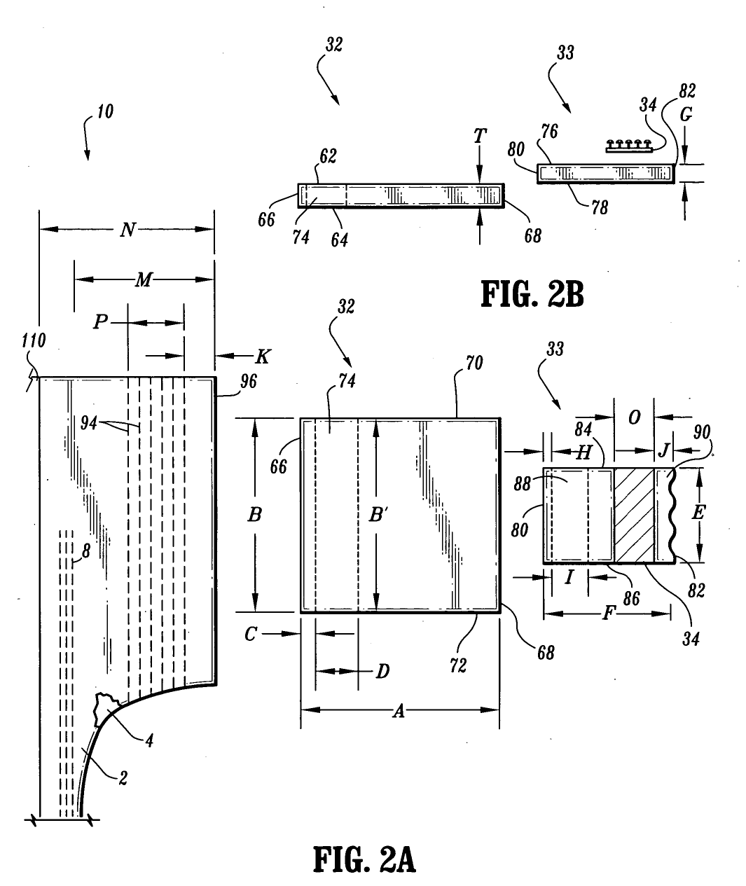

Referring to FIGS. 2A and 2B, the fastener 32 includes a rear edge or side 70 having a distance A, a front edge 72 of distance A, and a thickness T. Fastener 32 includes a top surface 62, a bottom surface 64, and has a proximal edge 66 and a distal edge 68 of a distance B. Fastener 32 includes a fastener attachment region 74 for attachment to the diaper chassis. Attachment region 74 is disposed a distance C from the proximal edge 66 of the fastener 32, and has a lateral and a longitudinal dimension of D and B′ respectively.

In an alternative embodiment, fastener 32 is a white elastomeric tri-laminate such as a Tredegar 306D, available from Tredegar Film Products of Richmond, Va. The respective dimensions A and B of fastener 32 are generally between 50-90 mm and preferably 70 mm. The lateral distance D of the fastener attachment region 74 is generally between 5-15 mm and preferably 10 mm, and the longitudinal distance B′ of fastener attachment region 74 is equal to or less than the distance B of the fastener 32. The distance C is generally between 1-4 mm and preferably 2 mm.

Still referring to FIGS. 2A and 2B, the closure tab 33 has a rear edge 84 and a front edge 86, each of a distance F. A proximal edge 80 and a distal edge 82 of the closure tab 33 are each of a distance E, and the thickness of closure tab 33 is a distance G. Closure tab 33 includes a top surface 76 and a bottom surface 78 A closure tab attachment region 88 for attachment to the fastener 32 is disposed a distance H from the proximal edge 80 of the closure tab 33. Closure tab attachment region 88 has a lateral dimension I and longitudinal dimension E. Mechanical closure material 34 is disposed near or at the distal edge 82 of the closure tab 33 and has lateral and longitudinal dimensions E and O respectively. Mechanical closure material 34 may include any hook-and-loop type material, adhesive, or other means that is capable of holding diaper 10 on a wearer. A region 90 for lifting or actuating the closure tab 33 is disposed near or at distal edge 82 and has a lateral dimension of J and a longitudinal dimension E. Edge 82 may be wavy, straight, or otherwise shaped. As more specifically described below, the closure tabs 33 may be formed with materials that are elastic, non-elastic or a combination thereof and may be attached to the fastener 32 by adhesive, ultrasonic, thermal bonding or the like.

In another embodiment, closure tab 33 is a white non-stretch bi-laminate such as Tredegar 100D available from Tredegar Film Products of Richmond, Va. In such embodiment, lateral dimension F of closure tab 33 is generally between 30-66 mm and preferably 46.5 mm. Longitudinal dimension E is generally between 20-50 mm and preferably 35 mm. Attachment region 88 is generally 1-4 mm and preferably 2 mm from proximal edge 80. The lateral dimension I of attachment region 88 is generally between 5-15 mm and preferably 10 mm. The longitudinal dimension of the attachment region 88 is generally equal to or less than that of the closure tab 33, that is, generally between 20-50 mm and preferably 35 mm In this illustrative embodiment, the mechanical closure material 34 may be, for example, a white hook element with 1400 pin density, such as item number CS600 available from 3M Corporation. The lateral dimension of the mechanical closure material 34 is generally between 10-20 mm and preferably 15 mm. The longitudinal dimension of the mechanical closure material 34 is generally equal to or less than that of the closure tab 33, that is, generally between 20-50 mm and preferably 35 mm.

With further reference to FIGS. 2A and 2B, diaper 10 includes backsheet 4 comprised of a soft white spunbond material which generally weighs between 15 and 35 grams and preferably 25 grams per square meter (gsm). In this embodiment, topsheet 2 may comprise, for example, a white spunbond material weighing generally between 10 and 20 grams and preferably 15 gsm. Backsheet 4 includes a plurality of edge glue lines 94 for adhering to the bottom surface 64 of fastener 32 along fastener attachment region 74. More specifically, glue lines 94 are disposed a distance K from a most distal edge 96 of diaper 10 and extend proximally along backsheet 4 for a distance P. Glue lines 94 may be formed of an adhesive, such as, for example, NS 34-5640 as available from National Starch Corporation. It is contemplated that backsheet 4 may include other connective means arranged in a variety of configurations, such as, for example, a plurality of zigzagged point bonds. Distal edge 96 of backsheet 4 is a distance M from an outermost leg elastic 8 of diaper 10. Distal edge 96 is a distance N from an edge of the central poly backsheet panel 110.

The distance K from the most distal edge 96 of the diaper 10 to the glue line 94 is generally 2-10 mm and preferably 5 mm. The lateral distance P is generally 10-20 mm and preferably 15 mm. In such embodiment, the distance M from the distal edge 96 of backsheet 4 to the outermost leg elastic 8 of diaper 10 is generally 20-40 mm and preferably 33 mm. The distance N from the most distal edge 96 to the edge of the central poly backsheet panel 110 is generally 25-75 mm and preferably 51 mm.

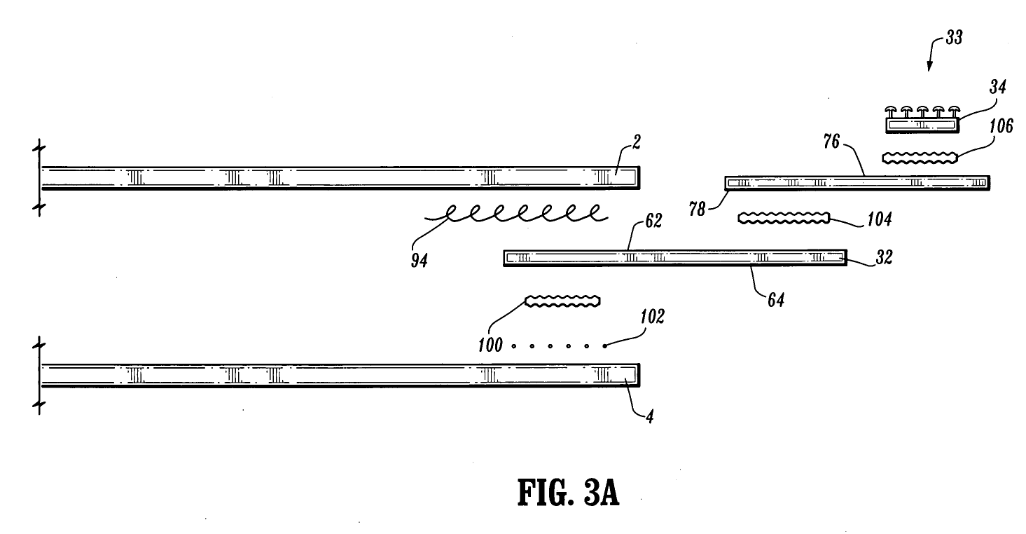

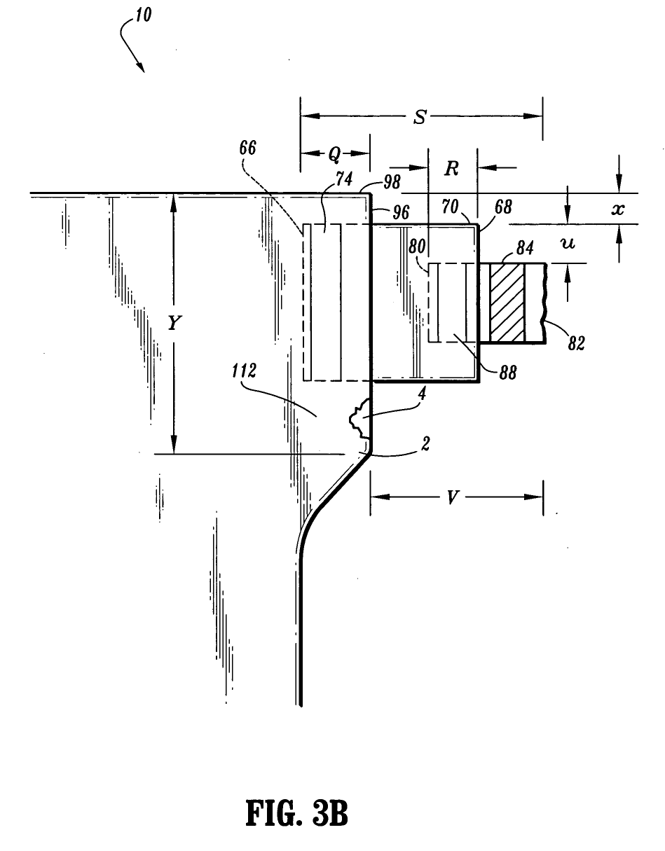

Referring to FIGS. 2A, 3A and 3B, fastener 32 is attached to the diaper chassis along a fastener attachment area 112. This fastener attachment area 112 is disposed generally along the most distal portion of diaper 10. More specifically, fastener attachment area 112 extends longitudinally from a rear edge 98 of the diaper 10 for a distance Y, and extends laterally from the distal edge 96 of the diaper 10 for a distance M. The fastener 32 is attached to the diaper 10 along attachment area 112 such that the proximal edge 66 of fastener 32 is disposed a distance Q from the distal edge 96 of diaper 10. In this way, the rear edge 70 of the fastener 32 is a distance X from a rear edge 98 of the diaper 10.

As shown in FIG. 3B, the closure tab 33 is attached to the fastener 32 such that the proximal edge 80 of closure tab 33 is disposed a distance R from the distal edge 68 of fastener 32. The closure tab 33 is substantially centered along the distal edge of fastener 32 such that the rear edge 84 of the closure tab 33 is a distance U from the rear edge 70 of the fastener 32. The proximal edge 66 of the fastener 32 is a distance S from the distal edge 82 of the closure tab 33. This distance S may represent the combined lateral dimension of the fastener 32 with closure tab 33 attached thereto. In this illustrative embodiment, the most distal edge 96 of diaper 10 is a distance V from the distal edge 82 of closure tab 33.

As shown in FIG. 3A, the bottom surface 64 of the fastener 32 is adhered along fastener attachment region 74 to the backsheet 4 by gluelines 94 thereon and by adhesive layer 100. Adhesive layer 100 may be, for example, an HL-1696-XZP adhesive, as available from H.B. Fuller Corporation. The top surface 62 of the fastener 32 is adhered to a distal end of the top sheet 2 by a construction adhesive layer 102. Adhesive layer 102 may be, for example, a control weave application. The bottom surface 78 of the closure tab 33 is adhered, along closure tab attachment region 88, to the top surface 62 of fastener 32 by an adhesive layers 104. Mechanical closure material 34 is adhered to the top surface 76 of the distal end of closure tab 33 by an adhesive layer 106. However, variations on the attachment location and configuration of diaper 10, fastener 32 and closure tab 33 will be apparent to those skilled in the art based on the teachings herein, and all such variations are within the scope of the present invention. For example, the diaper 10, fastener 32 and closure tab 33 may be attached to the each other by other connective means such as, for example, ultrasonic, thermal bonding, compression bonding or the like.

The diaper 10 and fastener 32 are attached such that the distance Q from proximal edge 66 of fastener 32 to the most distal edge 96 of diaper 10 is generally between 15-40 mm and preferably 25 mm. The distance R from the proximal edge 80 of closure tab 33 to the distal edge 68 of the fastener 32 is generally between 10-30 mm and preferably 20 mm. The distance S from the proximal edge 66 of the fastener 32 to the distal edge 82 of the closure tab 33 is generally between 80-150 mm, preferably 96.5 mm. The distance U from the rear edge 84 of the closure tab 33 to the rear edge 70 of the fastener 32 is generally between 10-30 mm and preferably 17 mm. The distance V from distal edge 96 of diaper 10 to the distal edge 82 of closure tab 33 is generally between 40-150 mm and preferably 71.5 mm. The distance X from the back edge 70 of the fastener 32 to the rear edge 98 of diaper 10 is generally between 5-30 mm and preferably 15 mm. The longitudinal dimension Y of the fastener attachment area 112 is generally between 75 and 125 mm and preferably 114 mm. The lateral dimension M of the fastener attachment area 112 is generally between 20-45 mm and preferably 33 mm. The length of the fastener 32 as measured in a longitudinal direction is between en 50-70% of the length of the fastener attachment area 112.

One advantage provided by the present invention is that the large stretch fastener 32 allows the chassis of diaper 10 to be smaller in size. For example, although the length L of diaper 10 in FIG. 1A is conventional, the overall width W of diaper 10 in FIG. 1A is reduced. The reduction in width W occurs along the distal ear portions 38, 46 of diaper 10 (since the core and leg gather section widths remain constant with the particular size of the diaper). This reduction in material provides for a more economical diaper.

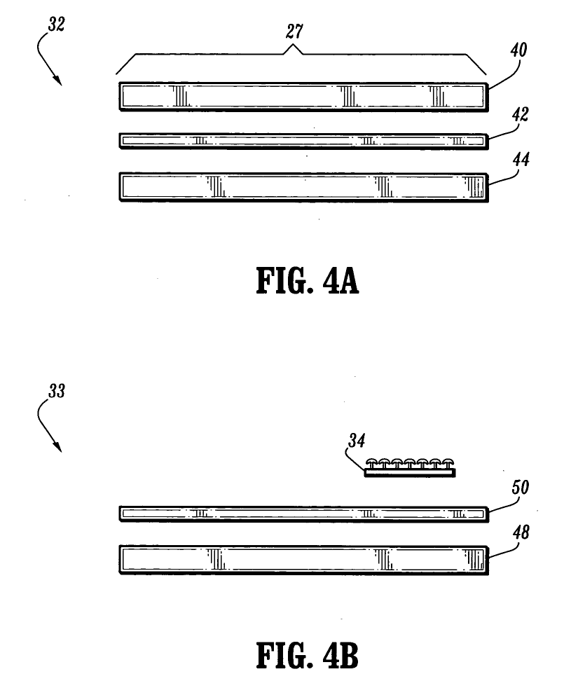

In the alternative embodiment that illustrates the elastomeric tri-laminate fastener 32, described above and as shown in FIG. 4A, stretchable zone 27 includes an extensible layer 42, and at least one non-woven layer 40 adjacent thereto. Stretchable zone 27 has extensible layer 42 disposed between a first non-woven layer 40 and a second non-woven layer 44. Non-extensible or deadened proximal and distal portions 23, 25 include layers 40, 42, 44. Fastener 32 may also include a layer of non-stretch or non-elastic film (not shown) adjacent layers 40, 42, 44.

Extensible layer 42 may be formed from, for example, a urethane based elastic or stretch film, such as EXTRAFLEX® available from Tredegar Film Products of Richmond, Va. Elastic layer 42 may also be formed from other types of elastic films, multidirectional elastic aggregate such as elastic webbing, netting, or scrim elastic, foam, strands or bands of suitable elastic materials, such as natural or synthetic rubber, spandex, LYCRA® and elastic polymers. Other suitable elastics will be apparent to those skilled in the art in light of the present disclosure.

The non-woven layers 40, 44 may be, for example, spunbonded polypropylene, polyethylene, or other non-woven material that encases elastic layer 42, protects the elastic layer 42, protects the wearer from uncomfortable exposure to the elastic layer 42 or provides other benefits. Other uses for the non-woven layers 40, 44 will be apparent to those skilled in the art based on the present disclosure.

The bonding between layers 40, 42, 44 of fastener 32 may be accomplished using joining methods known in the art, such as, for example, compression bonds, heat bonds, ultrasonic bonds, adhesives and the like, or combinations of different bonding methods. The construction of such elastic laminates is known in the art, and a skilled artisan will be able to provide a suitable elastic laminate or other elastic design for fastener 32 without undue experimentation.

In the alternative embodiment that illustrates the non-stretch bi-laminate closure tab 33 described above and as shown in FIG. 4B, closure tab 33 includes a non-woven layer 48 adjacent to a layer of non-stretch or non-elastic film 50. Non-woven layer 48 may be spunbonded polypropylene, polyethylene, or other non-woven material, and the bonding between layers 48 and 50 may be accomplished using joining methods known in the art.

The invention of the present disclosure may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A fastener adapted for use with an absorbent article having a topsheet and a backsheet, the fastener comprising:
a rectangular extensible portion configured to extend in a lateral direction such that the fastener extends distally from the absorbent article,
the extensible portion including a fastener attachment region for attachment along a distal edge of the absorbent article, the fastener attachment region being distal of a proximal edge of the extensible portion; and
a non-extensible closure tab portion that includes a closure tab attachment region for attachment along a distal edge of the extensible portion, the closure tab attachment region being distal from a proximal edge of the non-extensible portion, wherein
a proximal end of the fastener is disposed between and attached to the topsheet and the backsheet, the proximal end being attached to the backsheet by a fastener adhesive layer disposed between the fastener and the backsheet, the proximal end being attached to the topsheet by a construction adhesive between the topsheet and fastener.

2. A fastener as recited in claim 1, wherein the extensible portion includes a proximal deadened zone and a distal deadened zone.

3. A fastener as recited in claim 1, wherein the non-extensible closure tab portion is substantially centered along a distal edge of the extensible portion.

4. A fastener as recited in claim 1, wherein the non-extensible closure tab portion includes hook elements configured for attachment with the absorbent article.

5. A fastener as recited in claim 1, wherein the extensible portion is disposed between the absorbent article and the non-extensible portion.

6. A fastener as recited in claim 1, wherein the extensible portion has a greater surface area relative to the non-extensible portion.

7. An absorbent article adapted for use with a subject, the absorbent article comprising:
a backsheet, a topsheet and an absorbent core disposed therebetween, the absorbent article extending from a first end to a second end along a longitudinal length thereof;
a fastener attached to the first end, and being engageable with the second end for disposing the absorbent article about the subject,
the first end including a fastener attachment area for attaching the fastener thereto, wherein a length of the fastener is between 50-90% of a length of the fastener attachment area.

8. An absorbent article as recited in claim 7, further including a closure tab attached to the fastener for engaging the second end of the absorbent article, wherein the length of the closure tab is between 50 and 90 percent of the length of the fastener.

9. An absorbent article as recited in claim 7, wherein the length of the fastener attachment area is in the range of 100-130 mm.

10. An absorbent article as recited in claim 7, wherein the length of the fastener is in the range of 50-120 mm.

11. An absorbent article adapted for use with a subject, the absorbent article comprising:
a backsheet, a topsheet and an absorbent core disposed therebetween, the absorbent article extending from a first end to a second end along a longitudinal length thereof;
a rectangular fastener including a distal non-stretch end, a proximal non-stretch end, and a stretch section between the non-stretch ends,
a rectangular non-stretch closure tab having a proximal end and a distal end, and
mechanical closure means attached to the closure tab and being engageable with the second end for disposing the absorbent article about the subject,
the proximal non-stretch end of the fastener being attached to a distal edge of the first end such that the fastener is substantially centered along the distal edge,
the distal non-stretch end of the fastener being attached to the proximal end of the closure tab such that the closure tab is substantially centered along the distal non-stretch end of the fastener, and
the distal end of the closure tab attached to the mechanical closure means.

12. An absorbent article as recited in claim 11, wherein the proximal non-stretch end of the fastener overlaps the distal edge of the first end in the range of 20-30 mm.

13. An absorbent article as recited in claim 11, wherein the distal non-stretch end of the fastener overlaps the proximal end of the closure tab in the range of 10-30 mm.

14. An absorbent article as recited in claim 11, wherein the proximal non-stretch end of the fastener includes a region for attachment with the first end, the region for attachment having a width in the range of 5-20 mm.

15. An absorbent article as recited in claim 11, wherein the region for attachment is disposed from a distal edge of the fastener by a distance in the range of 1-4 mm.

16. An absorbent article adapted for use with a subject, the absorbent article comprising:
a backsheet, a topsheet and an absorbent core disposed therebetween, the absorbent article extending from a first end to a second end along a longitudinal length thereof;
an extensible fastener attached to a distal edge of the first end,
a non-extensible closure tab attached to the fastener, and
mechanical closure means attached to the closure tab and being engageable with the second end for disposing the absorbent article about the subject,
a proximal end of the fastener being disposed between and attached to the topsheet and the backsheet, the proximal end being attached to the backsheet by a fastener adhesive layer disposed between the fastener and backsheet, the proximal end being attached to the topsheet by a construction adhesive between the topsheet and fastener,
a distal end of the fastener being attached along its upper surface to a proximal end of the closure tab by a closure tab adhesive layer disposed between the fastener and closure tab,
a distal end of the closure tab being attached along its upper surface to the mechanical closure means by a closure means adhesive layer disposed between the closure tab and mechanical closure means.

17. An absorbent article as recited in claim 16, wherein an inner surface of the backsheet includes a plurality of glue lines along a distal edge thereof for facilitating attachment to the fastener.

18. An absorbent article as recited in claim 16, wherein the mechanical closure means is a white hook fastening element having a pin density in the range of 1200-1400.

19. An absorbent article as recited in claim 16, wherein the fastener is a rectangular white tri-laminate with deadened zones on each end, and edge lengths in the range of 50-90 mm.

20. An absorbent article as recited in claim 16, wherein the closure tab is a rectangular white bi-laminate including a non-woven layer adjacent a layer of non-elastic film.
